(19) United States
(12) Patent Application Publication
(10) Pub. No.: US 2019/0125648 A1
(43) Pub. Date: May 2, 2019

(54) COMPOSITION FOR PREVENTING, IMPROVING OR TREATING HAIR LOSS COMPRISING DEOXYCHOLIC ACID AS ACTIVE INGREDIENT

(71) Applicants: SAMSUNG LIFE PUBLIC WELFARE FOUNDATION, Seoul (KR); SOLSION BIOMEDICAL INC., Seoul (KR)

(72) Inventors: Hyeon Cheol GWON, Seoul (KR); Dong Youn LEE, Seoul (KR); Hyung Suk JANG, Seoul (KR); Min Hee KIM, Seoul (KR)

(21) Appl. No.: 16/093,997

(22) PCT Filed: Aug. 11, 2016
(86) PCT No.: PCT/KR2016/008859

§ 371 Date: Oct. 16, 2018

(51) Int. Cl. A61K 8/63; A61K 8/34; A61Q 7/00

(52) U.S. Cl. CPC A61K 8/63; A61Q 7/00; A61K 8/347

(57) ABSTRACT

The present invention provides a composition comprising deoxycholic acid as an effective ingredient for preventing, alleviating, or treating hair loss. Based on deoxycholic acid that promotes proliferation and growth of follicular cells to ultimately exert a hair regrowth effect, the composition of the present invention can be applied as a hair loss preventing agent, a hair growth agent or a hair restorer.

### TECHNICAL FIELD

The present invention relates to a composition comprising deoxycholic acid as an effective ingredient for preventing, alleviating or treating hair loss.

### BACKGROUND

Hair loss has been one of the greatest worries for someone with the recent increase of interest in appearance. Hair loss has been a concern, for the most part, in men in their 40's to 50's. Recently, alopecia has spread to youth in their 20's and 30's. An increasing population of women is also reported to suffer from hair loss nowadays. A variety of environmental factors including diet imbalance, harmful air, polluted drinking water, acidic rain, and stress aggravate hair loss in people at the present day. Therefore, many studies are ongoing for prevention and treatment of hair loss.

Each hair grows in cycles of various stages consisting of anagen in which hairs grow, catagen which signals the end of the active growth of a hair, with the regression of a hair bulb, Talogen in which the dermal papilla rests while a hair remains on the scalp, and exogen in which the dermal papilla starts to act or a new hair grows while an old hair is shed.

Anagen stage (2-7 years) is an active growth period of a hair and is divided into two stages of producing hair which grows outwards from the bulb into hair follicles; and generating hard keratins in the hair follicles. The hair continues to grow itself until the catagen stage.

Catagen stage (2-3 weeks) is a period during which hair growth ceases and the metabolism slow down while maintaining the shape of the hair. At this stage, keratin is not produced. The catagen stage accounts for 1% of total hair growth. At this stage, the hair bulb shrinks and is divided into dermal papilla, and is surrounded by hair follicles and travels upwards. The cell division is ceases.

Talogen stage (3 months) is a period during which the dermal papilla shrinks and the hair follicle gradually shrinks, and the hair root crawls upwards and falls out. It is the period of hair loss until the next stage of growth and lasts for 3 to 4 months.

For a normal person, hairs in the anagen stages are predominant over those in the other stages whereas people with alopecia have hairs mostly in the Talogen stage, with a phenomenon of hair loss visible to a naked eye. As the hair loss progresses, the period of the anagen stage is shortened, resulting in the miniaturization of the hair. Accordingly, allowing the hair follicle in the Talogen stage to rapidly proceed to the anagen stage and prolonging the anagen stage are important for treatment of hair loss.

There are two kinds of medications approved for hair growth promotion by the FDA: minoxidil (transdermal agent) and finasteride (oral agent). Minoxidil was developed as an oral antihypertensive vasodilator medication and is now used as a topical agent for treatment of androgenic alopecia because hypertrichosis was observed in patients administered therewith. When applied to the scalp, minoxidil, which is a pyrimidine derivative, acts as a vasodilator to topically increase blood flow to activate keratinocytes whereby the progress of hair loss can be delayed and hairs in a new anagen stage appear. Thanks to this effect, minoxidil is widely used as a medication for hair loss treatment. Acting to inhibit type II 5α-reductase with the resultant effect of preventing hair loss and promoting hair regrowth, finasteride is the first oral medication approved for treatment of androgenic alopecia by the FDA in 1997 and at present, is used worldwide by about 2.6 million persons.

However, minoxidil has side effects including weight gain, edema, increased heart rates, angina pectoris, dermatitis, and itching. For finasteride, male sexual dysfunction was reported as a clinical case. These medications are thus limited in application or patients themselves are reluctant to use the medications. For the reason, the interest of consumers in the prevention of hair loss and the promotion of hair regrowth has turned toward safe agents derived from natural materials and therefore, active studies therefor are ongoing.

Throughout the entire specification, many papers and patent documents are referenced and their citations are represented. The disclosures of cited papers and patent documents are entirely incorporated by reference into the present specification, and the level of the technical field within which the present invention falls and details of the present invention are explained more clearly.

### SUMMARY

### Technical Problem

Leading to the present invention, intensive and thorough research into the development of an agent for prevention, alleviation or treatment of hair loss, conducted by the present inventors, resulted in the finding that deoxycholic acid, which is a bile acid, promotes the growth and proliferation of follicular cells to ultimately exhibit a hair regrowth effect.

Accordingly, an object of the present invention is to provide a pharmaceutical composition for preventing, alleviating or treating hair loss.

Another object of the present invention is to provide a quasi-drug composition for preventing or alleviating hair loss.

A further object of the present invention is to provide a cosmetic composition for preventing or alleviating hair loss.

Other purposes and advantages of the present invention will become clarified by the following detailed description of the invention, claims, and drawings.

### Technical Solution

According to one aspect thereof, the present invention provides a pharmaceutical composition comprising deoxycholic acid as an effective ingredient for preventing, alleviating or treating hair loss.

According to another aspect thereof, the present invention provides a quasi-drug composition comprising deoxycholic acid as an effective ingredient for preventing or alleviating hair loss.

According to a further aspect thereof, the present invention provides a cosmetic composition comprising deoxycholic acid as an effective ingredient for preventing or alleviating hair loss.

The present inventors have conducted research to develop an agent for treatment, alleviation, or treatment of hair loss. As a result, deoxycholic acid, a bile acid, is found to promote the growth and proliferation of follicular cells, thus ultimately exerting a hair regrowth effect.

Deoxycholic acid, which is used as the effective ingredient of the present invention, is a representative secondary bile acid, represented by the empirical formula C24H40O4, which is derived from cholic acid. Herein, deoxycholic acid and its salts are construed to fall within the scope of deoxycholic acid.

As used herein, the term “hair loss” refers to a loss of hair from the scalp or to hair thinning and can be interchangeable with alopecia. Examples of the hair loss include male-pattern hair loss (androgenic hair loss), alopecia areata, Talogen effluvium, drug-induced alopecia, mechanical alopecia, alopecia caused by a skin disorder, and cicatrical alopecia.

Therefore, the term “hair loss” should be understood to encompass all symptoms classified as hair loss in the art irrespective of whether the cause is direct or indirect.

According to an embodiment, the composition of the present invention is used for preventing hair loss or promoting hair growth or regrowth.

The term “preventing hair loss” means pertaining to preventing and suppressing alopecia as described above, the term “hair growth” means pertaining to prolonging the life of hair in the anagen stage to allow hairs to appear thick and healthy, and the term “hair regrowth” means pertaining to inducing or promoting the generation of hair in a new anagen stage. Therefore, the composition of the present invention may be used as a hair loss preventing agent, a hair growth agent, and/or a hair restorer.

The composition of the present invention may be formulated into suitable forms according to product modalities (pharmaceutical composition, quasi-drug composition, or cosmetic composition).

For example, the composition of the present invention may be in a form selected from the group consisting of a tonic, a spray, a cream, a lotion, an aerosol, oil, a solution, a suspension, a gel, an ointment, a paste, an emulsion, a liniment, a tablet, a capsule, a powder and a granule.

According to an embodiment, the composition of the present invention is an external skin preparation.

The composition of the present invention may comprise a different effective ingredient in addition to deoxycholic acid.

According to an embodiment, the different effective ingredient contained in the composition of the present invention is polyphenol.

The polyphenol available for the present invention may be exemplified by resveratrol, protocatechuic acid, gallic acid, p-hydroxybenzoic acid, caffeic acid, chlorogenic acid, cuomaric acid, ferulic acid, sinapic acid, pelargonidin, peonidin, delphinidin, malvidin, quercetin, myricetin, apigenin, luteolin, hesperetin, naringenin, eriodictyol, daidzein, genistein, glycitein, catechin and epicatechin.

According to a more particular embodiment, the polyphenol is resveratrol.

According to some embodiments, the composition of the present invention comprises 0.1-10% by weight of deoxycholic acid.

The pharmaceutical composition of the present invention may comprise a pharmaceutically acceptable carrier.

The pharmaceutically acceptable carrier may be one typically utilized for formulation and examples thereof include, but are limited to, lactose, dextrose, sucrose, sorbitol, mannitol, starch, acacia gum, calcium phosphate, alginate, gelatin, calcium silicate, microcrystalline cellulose, polyvinylpyrrolidone, water, syrup, methyl cellulose, methylhydroxybenzoate, propylhydroxybenzoate, talc, magnesium stearate and mineral oil.

In addition to the above-mentioned ingredient, the pharmaceutical composition of the present invention may further comprise a lubricant, a humectant, a sweetener, a flavoring agent, an emulsifier, a suspending agent, a preservative, etc.

For concrete suitable pharmaceutically acceptable carriers and agents, reference may be made to Remington's Pharmaceutical Sciences (19th ed., 1995).

The pharmaceutical composition of the present invention may be administered orally or parenterally (e.g., transdermally).

The suitable dose of the pharmaceutical composition of the present invention may vary depending on various factors including the formulation method, the patient's age, weight, sex, the period of hair loss, the state of hair loss, the time of administration, the route of administration, excretion rate, and response sensitivity and a skilled physician with a moderate level of experience can easily determine and prescribe an effective dose for the desired prevention or treatment.

Additionally, the pharmaceutical composition may be prepared in a unit dose form or prepared to be contained in a multi-dose container by formulating with a pharmaceutically acceptable carrier and/or excipient, according to a method that a person skilled in the art can easily perform. In particular, the formulation may be in the form of a solution in oil or aqueous medium, a suspension, or an emulsion, or an extract, powders, granules, tablets, or capsules and may further include a dispersing agent or stabilizer.

According to the Korean Pharmaceutical Affairs Act, a quasi-drug refers to a product that has a weak action on the human body or does not directly act on the human body and which is not a tool or a machine, but an analogue thereof. A notice from the ministry of health and welfare in Korea classifies an external use agent for prevention of hair loss or promotion of hair regrowth as a quasi-drug.

In addition, according to the Korean cosmetics act, a cosmetic refers is defined as a product that cleans or beautifies the human body to enhance the charm or appearance of a person or is applied to the human body to maintain or enhance the health of the skin or hair and which has a slight effect on the human body.

The quasi-drug or cosmetic composition of the present invention may be prepared into any formulation form typically used in the art, for example, various external use agents such as paste, mousse, gel, liquid, emulsion, suspension, cream, ointment, sheet, aerosol, spray and liniment.

According to an embodiment, the quasi-drug or cosmetic composition of the present invention may be in the form of a hair shampoo, a hair rinse, a hair tonic, a hair conditioner, a hair essence, a hair lotion, a hair nutrient lotion, a hair cream, a hair nutrient cream, a hair moisturizing cream, a hair massage cream, a hair wax, a hair aerosol, a hair pack, a hair soap, a hair gel, a hair glaze, a hair mousse or a hair spray.

### Advantageous Effects

Features and advantages of the present invention are summarized as follows:

(a) The present invention provides composition comprising deoxycholic acid as an effective ingredient for preventing, alleviation, or treating hair loss.

(b) Because deoxychloic acid promotes the proliferation and growth of follicular cells to ultimately exhibit the effect of hair regrowth, the composition of the present invention can be used as a hair loss preventer, hair growth agent or hair restorer.

### BRIEF DESCRIPTION OF THE DRAWINGS

FIG. 1 shows changes in the area of hair loss of group T (testosterone 10 mg/ml), group TD (testosterone 10 mg/ml+deoxycholic acid 1 mg/ml), and group TF (testosterone 10 mg/ml+finasteride 1 mg/ml).

FIG. 2 shows counts of follicular cells in group T (testosterone 10 mg/ml), group TD (testosterone 10 mg/ml+deoxycholic acid 1 mg/ml), and group TF (testosterone 10 mg/ml+finasteride 1 mg/ml).

FIG. 3 shows changes in the area of hair loss of group T (testosterone 10 mg/ml), group TR (testosterone 10 mg/ml+resveratrol 1 mg/ml), group TF (testosterone 10 mg/ml+finasteride 1 mg/ml), and group TRF (testosterone 10 mg/ml+resveratrol 1 mg/ml+finasteride 1 mg/ml).

### DETAILED DESCRIPTION

Hereinafter, the present invention will be described in detail with reference to examples. These examples are only for illustrating the present invention more specifically, and it will be apparent to those skilled in the art that the scope of the present invention is not limited by these examples.

### EXAMPLES

### Experimental Materials and Methods

### Experimental Materials

Testosterone was purchased from Wako Pure Chemical Industry, and deoxycholic acid, finasteride, resveratrol, and N-Methyl-2-pyrrolidone (NMP) were Sigma Aldrich. Ethanol and glycerol was purchased from OCI Company Ltd. and Biosesang Company Ltd., respectively.

Animal Preparation

B6CBAF1/j hybrid mice (5 weeks old, male, Charles River Japan) were acclimated for one week in a breeding room and then used from the time of 6 weeks after birth.

### Preparation of Agent

For use in an injection agent, a solvent was prepared to contain 15% of ethanol, 5% of NMP, and 80% of PBS. Solutions were prepared by mixing the solvent with the following solutes: group T (testosterone 10 mg/ml), group TD (testosterone 10 mg/ml+deoxycholic acid 1 mg/ml), group TR (testosterone 10 mg/ml+resveratrol 1 mg/ml), group TF (testosterone 10 mg/ml+finasteride 1 mg/ml), and group TRF (testosterone 10 mg/ml+resveratrol 1 mg/ml+finasteride 1 mg/ml).

### Animal Test

The animals were anesthetized with isoflurane through inhalation and injected subcutaneously at the neck with 100 μl of a testosterone solution (10 mg/ml) every day. From a couple of weeks, they were photographed along with a scale bar to quantify a hair loss area. After six weeks of testing, the mice were sacrificed by means of a CO₂ incubator. Test areas of the skin were taken, fixed with formalin, and prepared into paraffin blocks. The blocks were horizontally sectioned and dyed with H&E (Hematoxylin & Eosin) to count follicles per mm².

### Test Result

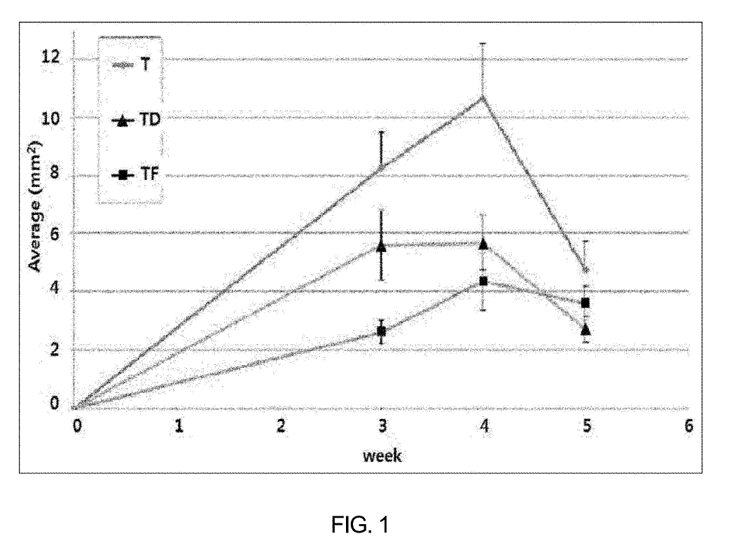

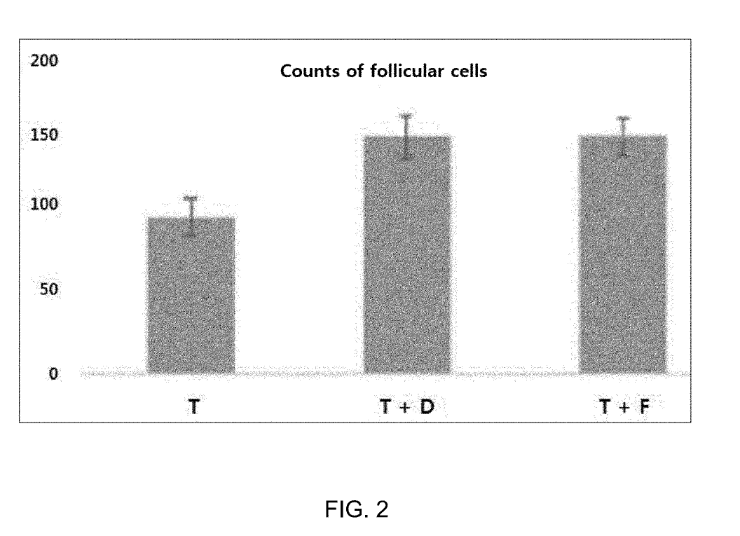

As shown in FIG. 1, the deoxycholic acid-treated group (group TD) was observed to remarkably reduce in hair loss area from week 3 after drug treatment, compared to the group treated with testosterone alone (group T). On week 4, a similar effect was found between the deoxycholic acid-treated group (group TD) and the finasteride-treated group (group TF) (FIG. 1). In addition, it can be seen in FIG. 2 that remarkable proliferation and growth of follicular cells were detected in the deoxycholic acid-treated group (group TD), compared to the group treated with testosterone alone (group T).

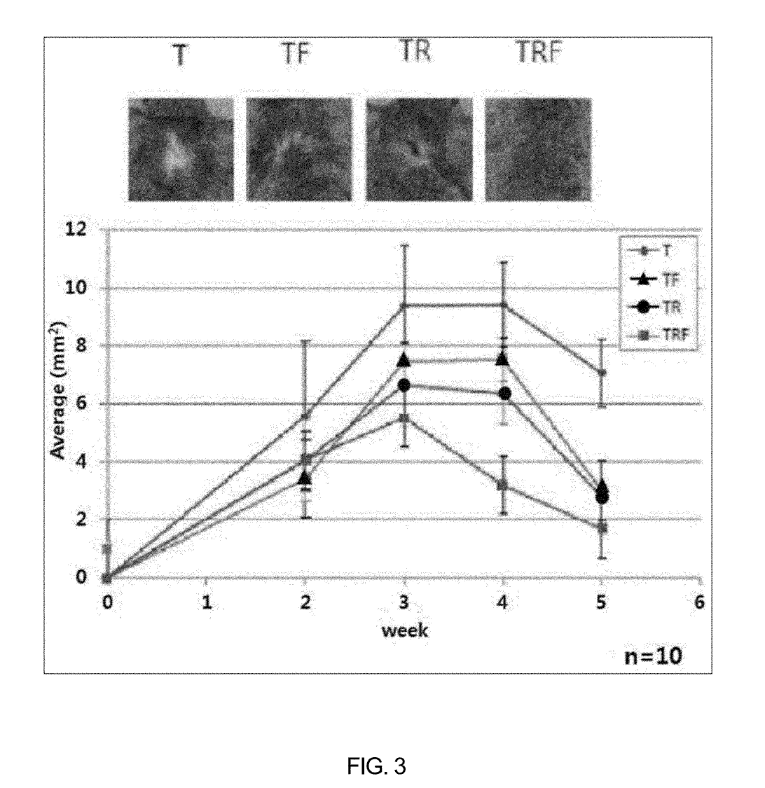

Even after two weeks, as shown in FIG. 3, the hair loss continued to increase in the group treated with testosterone alone (group T) while an increment in hair loss area was greatly reduced in the group treated with resveratrol (group TR) and with resveratrol and finasteride in combination (group TRF) (FIG. 3).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A composition comprising:
deoxycholic acid; and acceptable carrier.

2.-3. (canceled)

4. The composition of claim 1, wherein the composition is used for preventing hair loss or promoting hair growth or hair regrowth.

5. The composition of claim 1, wherein the composition is an external use agent.

6. The composition of claim 1, wherein the composition has a form selected from the group consisting of a tonic, a spray, a cream, a lotion, an aerosol, an oil, a solution, a suspension, a gel, an ointment, a paste, an emulsion, a liniment, a tablet, a capsule, a powder, and a granule.

7. The composition of claim 1, wherein the deoxycholic acid promotes proliferation or growth of follicular cells.

8. The composition of claim 1, wherein the composition further comprises polyphenol as an effective ingredient.

9. The composition of claim 8, wherein the polyphenol is resveratrol.

10. A method for preventing or alleviating hair loss comprising:
administering to a subject a composition comprising deoxycholic acid as an effective ingredient.
